(19) United States
(12) Patent Application Publication
(10) Pub. No.: US 2006/0009435 A1
(43) Pub. Date: Jan. 12, 2006

(54) Synthesis and powder preparation of fluticasone propionate

(76) Inventors: Joseph Kaspi, Givatayim (IL); Oded Arad, Rechovot (IL); Michael Brand, RaAnana (IL); Moty Shookrun, Petach-Tikva (IL); Simona Malka, Beer-Sheba (IL); Mohammed Alnabari, Hura (IL); Shalom Hazan, Beer-Sheva (IL); Vlado Malesevic, Beer-Sheva (IL)

Correspondence Address: Martin D. Moynihan;PRTSI, Inc., P. O. Box 16446, Arlington, VA, 22215, US

(21) Appl. No.: 11/159,241

(22) Filed: Jun. 23, 2005

Related U.S. Application Data
(60) Provisional application No. 60/581,702, filed on Jun. 23, 2004.
(60) Provisional application No. 60/623,877, filed on Nov. 2, 2004.

(51) Int. Cl. A61K 31/573; A61K 9/14; A61L 9/04

(52) U.S. Cl. 514/179; 424/46

(57) ABSTRACT

An improved process for preparing fluticasone propionate, performed in the presence of water, is disclosed. Further disclosed is a process for preparing a fluticasone propionate that is highly suitable for administration by inhalation. Further disclosed are fluticasone propionate and a powdered fluticasone propionate prepared by these processes and pharmaceutical compositions for administration by inhalation containing same. A process of purifying a key intermediate in the synthesis of fluticasone propionate is also disclosed.

### RELATED PATENT APPLICATIONS

This application claims the benefit of U.S. Provisional Patent Application No. 60/581,702, filed Jun. 23, 2004, and of U.S. Provisional Patent Application No. 60/623,877, filed Nov. 2, 2004, the teachings of which are incorporated herein by reference in their entirety.

### FIELD AND BACKGROUND OF THE INVENTION

The present invention relates to an improved process of preparing fluticasone propionate. The present invention further relates to a process of preparing a dry powder form of fluticasone propionate, which is highly suitable for pharmaceutical formulations.

(S-fluoromethyl-6α,9α-difluoro-11β-hydroxy-16α-methyl-3-oxo-17α-propionyl oxyandrosta-1,4-diene-17β-carbothioate), also known and referred to herein and in the art as fluticasone propionate, is a steroidal anti-inflammatory agent of the glucocorticoid family. Fluticasone propionate is a synthetic corticosteroid which is related to the naturally-occurring steroid hormone cortisol (hydrocortisone), produced by the adrenal glands. Fluticasone propionate is known as a potent agent for the treatment of inflammatory respiratory disorders such as asthma, perennial rhinitis and of topical inflammatory conditions.

Fluticasone propionate is marketed worldwide under brand names such as Flovent™, Advair Diskus™, Flonase™, Cutivate™, Atemur™, Flutide™, Flutivate™ and Viani™.

This compound was first disclosed in U.S. Pat. No. 4,335,121. According to the teachings of this patent, fluticasone propionate is prepared via a multi-step process, which is highly inefficient, resulting in about 50% yield. The process taught in this patent is further limited by the use of expensive reagents such as silver fluoride, which is used for halide exchange from chloride to fluoride, and cumbersome conditions such as dark environment, which renders it inadequate for a preparation in commercial scale.

U.S. Patent Application having the Publication No. 2004/0116396 and Israeli Patent IL 109,656, which are incorporated by reference as if fully set forth herein, teach an improved process of preparing fluticasone propionate. According to the teachings of these documents, fluticasone propionate can be obtained by the direct esterification of the thiocarboxylic acid (6S,9R,10S,11S,13S,16R,17R)-6,9-difluoro-11-hydroxy-10,13,16-trimethyl-3-oxo-17-(propionyloxy)-6,7,8,9,10,11,12,13, 14,15,16,17-dodecahydro-3H-cyclopenta[a]phenanthrene-17-carbothioic S-acid (Compound I), with a halofluoromethane, such as chlorofluoromethane, bromofluoromethane and iodofluoromethane, in the presence of a base, and optionally in the presence of a phase transfer catalyst such as tetrabutylammonium bromide, in an appropriate organic solvent, as is presented in Scheme 1 below.

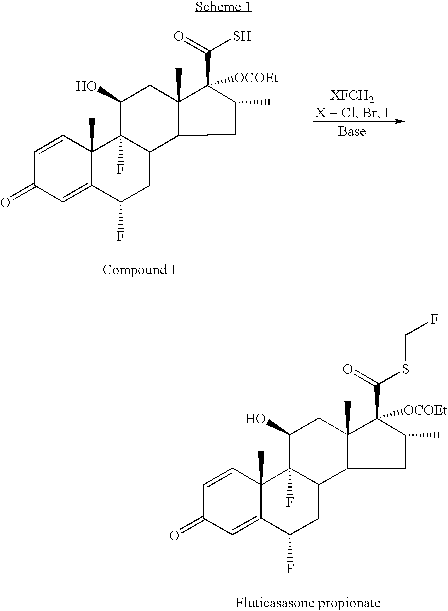

This process, however, is highly disadvantageous since the product is obtained in poor yields of 55-60%. In addition, the purity of the obtained product is relatively low and inadequate for pharmaceutical use. The obtained product contains a substantial amount of impurities, which is higher than the allowed level of impurities for a pharmaceutical product. Thus, additional laborious and costly purification steps are required in order to provide a product that has a pharmaceutically acceptable level of impurity.

There is thus a widely recognized need for, and it would be highly advantageous to have, an improved process of preparing fluticasone propionate in high yields and purity, which is safe, efficient and applicable to large scale manufacture.

As is mentioned hereinabove, fluticasone propionate is mainly used in the treatment of inflammatory respiratory disorders such as asthma. Fluticasone propionate is therefore aimed at entering the respiratory tract and reducing the inflammation which causes spasms that narrow the airways.

As a drug which is mainly used for the treatment of respiratory disorders, the typical mode of administration of fluticasone propionate is by inhalation. Administration by inhalation is most commonly affected by devices such as dry powder inhalers (DPI) and metered-dose inhalers (MDI) and mostly involves administration of a powdered form of the drug.

Thus, corticosteroids such as fluticasone propionate, as well as other drugs formulated and intended for administration by inhalation for pulmonary treatment are required to have a particular particle size and particle size distribution, in order to obtain an effective therapeutic activity. On the one hand particles should be small enough to penetrate the lungs, since inadequately large particles will not reach their target bodily sites and cavities, and on the other hand too small particles are not desired since they deliver a suboptimal local dosage which will not treat the condition effectively at that site.

Typically, the desired particles size of a drug for inhalation is about 1-10 microns, and even 1-5 microns, whereby the desired particles size distribution is such that minimal amounts of particles sized below 1 micron and above 5 microns are present. Such a particle size distribution can be achieved by mechanical pulverization techniques such as milling and micronization. As is well known in the art, milling is a collective term used to describe solid pulverization techniques which typically afford relatively large particles sizes, e.g., greater than 10 microns, whereby micronization is a collective term used to describe solid pulverization techniques which typically afford relatively small particles size. Apart from the particles size, other desired characteristics of a dry powder, delineated hereinafter, are also affected by the drying and milling or micronization processes.

When formulated for administration by inhalation as dry powders, drugs should further have additional characteristics which are extremely important for their efficient therapeutic use. These include, for example, shape, morphology, surface properties and electrostatic charge. The particles shape can be polygonal, cylindrical, spherical or oval; the morphology can be amorphous or crystalline; the surface of the particles can be smooth or rough, and accordingly has lower or higher area. The particles may further carry an electrostatic charge which stems from the milling and/or micronizing technique, chemical properties of the particular milling and/or micronizing substrate and environmental conditions.

The shape of the particles affects two major traits which are important in dry powder modes of therapeutic administration: dry powder flow and the tendency for agglomeration, wherein the powder flow is beneficial and the tendency for agglomeration is detrimental. The free flowing and tendency for agglomeration characteristics of a substance, as well as the surface area thereof, are oftentimes affected by the surface morphology of the particles.

Thus, when intended for use in the treatment of respiratory disorders, fluticasone propionate in a dry powder form desirably has the following characteristics: high purity, a crystalline form, a well defined, narrow particle size distribution ranging essentially between 1-5 microns, and free flowing with minimal tendency to aggregate to larger particles. In addition, spherical particles are preferred due to the roughness of their surface, which leads to increased separation space between particles, thus preventing agglomeration. Particles devoid of an electrostatic charge are further preferred since such a charge may affect the tendency for agglomeration and may also present safety hazards and difficulties in the packaging process in bulk manufacturing scale.

While the desired particles size of fluticasone propionate and other drugs that are intended for administration by inhalation can be achieved by micronization, this process typically generates particles having a substantially amorphous surface. Amorphous substances are typically disadvantageous due to the relatively high susceptibility thereof to unwanted moisture absorption, which may affect their surface area and free following characteristics, in comparison to crystalline substances.

In addition, the effectiveness of the micronization process is sensitive to the hardness of the crystals and therefore it may be difficult to reduce the particle size of some substances below a certain size. Attempts to further reduce the particle size in such cases will typically result in broadening of the particles size distribution due to the formation of more hyperfine particle instead of reduction of the median diameter.

The most widely used milling and/or micronization techniques, when applied on drugs, are typically associated with a rather limited ability to control the abovementioned product characteristics (Malcolmson and Embleton, Pharm. Sci. Technol., (1998), 1,394-398) and sometimes pose other limitations on the formulation process of drugs.

For example, common mechanical milling and micronization processes oftentimes involve physical contact of the crude drug with metallic or polymeric objects in order to achieve the reduction of particle size. This contact, which involves abrupt heat generation due to friction may promote and enhance chemical reaction between the drug and ambient chemicals, such as oxygen and the drug itself, which may lead to chemical modifications of the drug during and after the milling process (Kaneniwa and Ikekawa, Chem. Pharm. Bull., (1972), 20, 1536-1543).

Furthermore, the requirement of a narrow distribution of particle size is difficult or impossible to achieve with mechanical milling techniques. The most common milling technique used for obtaining particles having an average size in the range of 1-10 microns is air jet milling. However, this technique does not allow sufficient control of the abovementioned product characteristics (Malcolmson and Embleton, Pharm. Sci. Technol., (1998), 1,394-398). In addition, particles micronized by air jet milled exhibit a broad particle size distribution (Muller et al., Control Rel. Bioact. Mater. (1996), 22, 574-575). The typical broad particle size distribution of an air jet milled powder is caused by the need to keep the milling process going until the largest particles fall within the maximum size requirements while the particles which already reached that size are excessively milled.

The surface morphology of a mechanically micronized crystalline particle is also difficult, and sometimes impossible, to control. When a direct mechanical force is applied on a large crystalline particle, the micronization is controlled by crystal cleavage. Crystal cleavage is defined as a smooth break along the plane of a lattice layer which produces a flat smooth face. Thus, crystal cleavage typically occurs at the crystal face with the lowest attachment energy, i.e., the most brittle direction of the lattice (Roberts, et al. J. Mater. Sci. (1994), 29, 2289-2296). The high-energy input required to reduce the particle size against the relative high crystal lattice free energy (Ogura and Sobue, J. Appl. Polymer Sci., (1970), 14, 1390-1393), substantially reduces the efficiency of the mechanical micronization process (Parrott, Encyclopedia of pharmaceutical technology, 1990, vol. 3, 101-121). In addition, a flat-flake or elongated-rod shaped particles with high tendency to agglomerate and clog are typically obtained.

The use of the presently common mechanical milling processes for obtaining drug powders is further limited by its adverse effect on other physical properties of the formed particles. Mechanical milling processes oftentimes lead to the formation of a thermodynamically activated surface, and thus alters the surface properties and, as a result, the physical properties of the drug. Thus, for example, crystalline solid surfaces are typically uncontrollably converted to partially amorphous (disordered) surfaces during the milling process. The resulting disordered surface adversely affects properties such as the free flowing of the powders. In addition, common mechanical milling processes may result in particles with higher and irregular surface area, which are further characterized by a higher tendency to accumulate electrostatic charge, as a result of the mechanical friction and morphology of the particles. Electrostatically charged powders typically exhibit poorer flow properties and high tendency for agglomeration due to high particulate cohesion forces (Mackin et al., Int. J. Pharm. (2002), 231, 213-226). In view of the limitations associated with mechanical milling and/or micronization processes, alternative techniques for preparing a dry powder form of a drug, which would be suitable for treating respiratory disorders, have been developed.

U.S. Pat. No. 6,406,718, for example, describes a process for the preparation of fluticasone propionate having a specific particle size distribution and specific dynamic bulk density properties. The process taught in this patent involves a special technique that utilizes supercritical fluids, and the product is obtained as a novel crystalline form. This process, however, is limited by its high operational costs and complexity, and is further limited by inefficient control of some of the important properties mentioned hereinabove, e.g., parcel morphology and uniformity.

U.S. Patent Application having the publication No. 2004/0081626 describes a process for producing powders of pharmaceutically active agents, including fluticasone propionate, suitable for administration by inhalation. According to the teachings of this patent application, the drug powder is obtained as crystalline spherical particles. The process is effected by providing a solution of the drug in a liquid carrier, followed by atomizing the liquid carrier into droplets by spraying and suspending the droplets while heat drying is effected. According to the teachings of U.S. Application 2004/0081626, the process clearly differs from conventional spray drying processes since the atomization of the feedstock is performed in a separate part of the device and the mist flows into a heating chamber where the solvent in the droplets is vaporized, while in the conventional spray drying device the atomization and heating is afforded by the same factor which is the hot gas stream aiding in the atomization stage, suspend the mist, heats and evaporate the solvent in one chamber. This process is therefore limited by complexity of the technique utilized thereby and further by the continuous exposure of the drug to heat throughout the drying process.

U.S. Patent Application having the publication No. 2001/0046474 and WO 99/16419 both describe a process for producing powders of pharmaceutically active agents, including fluticasone propionate, suitable for administration by inhalation. These patent applications teach the preparation of powders having hollow porous micro-spherical particles by providing a solution of the drug in a liquid carrier, atomizing the liquid carrier solution into droplets, suspending the droplets and spray-drying the resulting emulsion in the presence of phospholipid surfactants. Again, this process is limited by its complexity and is further disadvantageous due to the presence of phospholipid surfactants during the spray-drying process. Such additives are hard to remove from the final product and their presence may affect the purity of the final product.

WO 01/58425 describes a process for producing powders of pharmaceutically active agents, including fluticasone propionate, suitable for administration by inhalation. The process taught in this application involves dissolving the drug in water, adding a vinyl polymer such as poly(vinyl)alcohol (PVA) to the aqueous solution and spray-drying the solution by conventional spray-drying techniques. Again, this process is disadvantageous due to the presence of an additive such as PVA, which may affect the pharmaceutical purity of the final product and/or requires the use of larger amounts of the final product, such that overall using such an additive increases the cost of the drug's production and formulation.

U.S. Pat. No. 6,221,398 describes a process for producing powders of pharmaceutically active agents, including fluticasone propionate, suitable for administration by inhalation. According to the teachings of this patent, powders that contain crystalline particles of a drug are produced by dissolving the drug in a liquid solvent and transferring the solution as a jet stream into an anti-solvent, which is miscible with the solvent, followed by spray drying. This process thus requires a complicated technique, which utilizes a complexed solvent system and is therefore disadvantageous for use in an industrial scale.

WO 98/29096 describes a process for producing powders of pharmaceutically active agents, including fluticasone propionate, suitable for administration by inhalation. The process taught in this application is effected by dissolving the drug in acetone or ethanol, followed by the addition of an aqueous solution of lactose and simultaneous spray-drying of the combined solutions. This process is disadvantageous due to the presence of an additive such as lactose, which requires the use of larger amounts of the final product, and overall leads to increased cost of the drug's production and formulation.

In a recently published article (H. Steckel, N. Rasenack, P. Villax and B. W. Muller, International Journal of Pharmaceutics, 258, (2003), 65-75), a process for the preparation of fluticasone propionate that have special particle size distribution in the micron region is described. According to the teachings of this publication, such particles are obtained by dissolving the substance in acetone and precipitating it by a solvent change method in the presence of cellulose ether, such as hydroxypropylmethyl-cellulose, as a stabilizing hydrocolloid (gel). By rapidly pouring the drug solution into the polymer-rich water phase, the previously molecularly dispersed drug is associated to small particles and is simultaneously stabilized against crystal growth by the hydrophilic polymer. The resulting dispersion is then spray dried to give solid particles of fluticasone propionate. Still, this process involves a complicated technology and is further limited by the use of a substance that may affect the pharmaceutical purity of the final product and/or requires the use of larger amounts of the final product, such that overall using such a technology results in cost-inefficiency of the drug's production and formulation.

In summary, the presently known methods and techniques for obtaining fluticasone propionate which is suitable for administration by inhalation, and thus can be used in the treatment of disorders in the respiratory tract, are limited either by the physical characteristics of the obtained product, by using expensive and complicated machinery, techniques and chemicals, and/or by mixing the pure substance with additives.

There is thus a widely recognized need for, and it would be highly advantageous to have, a process for obtaining a dry powder form of fluticasone propionate suitable for administration by inhalation, devoid of the above limitations.

### SUMMARY OF THE INVENTION

The present inventors have now surprisingly found that fluticasone propionate can be efficiently prepared by reacting the thiocarboxylic acid, Compound I with a halofluoromethane, in the presence of water. The present inventors have further found, surprisingly, that a powdered fluticasone propionate, which is highly suitable for administration by inhalation can be obtained using a conventional spray drying technique, while avoiding the use of additives.

Thus, according to one aspect of the present invention there is provided a process of preparing S-fluoromethyl-6α,9α-difluoro-11β-hydroxy-16α-methyl-3-oxo-17α-propionyloxyandrosta-1,4-diene-17β-carbothioate (fluticasone propionate), which comprises: providing 6α,9α-difluoro-11β-hydroxy-17α-propionyloxy-16α-methyl-pregna-3-oxo-1,4-diene-17β-thiocarboxylic acid; reacting the 6α,9α-difluoro-11β-hydroxy-17α-propionyloxy-16α-methyl-pregna-3-oxo-1,4-diene-17β-thiocarboxylic acid with a halofluoromethane in the presence of an organic solvent, water and a base, to thereby obtain a reaction mixture containing fluticasone propionate; and isolating the fluticasone propionate from the reaction mixture, thereby obtaining the fluticasone propionate.

According to further features in preferred embodiments of the invention described below, the halofluoromethane is selected from the group consisting of chlorofluoromethane, bromofluoromethane and iodofluoromethane.

According to still further features in the described preferred embodiments the organic solvent is selected from the group consisting of tetrahydrofuran, 2-methyltetrahydrofurane, acetonitrile and any mixture thereof.

According to still further features in the described preferred embodiments the base is a tertiary alkylamine.

According to still further features in the described preferred embodiments the amount of the water ranges from about 1 weight percent to about 200 weight percents, more preferably from about 40 weight percents to about 70 weight percents, of the weight of the 6α,9α-difluoro-11 β-hydroxy-17α-propionyloxy-16α-methyl-pregna-3-oxo-1,4-diene-17β-thiocarboxylic acid.

According to still further features in the described preferred embodiments the process further comprises, prior to the reacting, purifying the 6α,9α-difluoro-11β-hydroxy-17α-propionyloxy-16α-methyl-pregna-3-oxo-1,4-diene-17β-thiocarboxylic acid.

According to still further features in the described preferred embodiments the purifying the 6α,9α-difluoro-11β-hydroxy-17β-propionyloxy-16α-methyl-pregna-3-oxo-1,4-diene-17β-thiocarboxylic acid comprises:
providing a solution of the 6α,9α-difluoro-11β-hydroxy-17α-propionyloxy-16α-methyl-pregna-3-oxo-1,4-diene-17′-thiocarboxylic acid and an organic solvent;
contacting the solution with an aqueous solution containing a base, to thereby provide an aqueous solution containing a base addition salt of the 6α,9α-difluoro-11β-hydroxy-17α-propionyloxy-16α-methyl-pregna-3-oxo-1,4-diene-17α-thiocarboxylic acid;
isolating the aqueous solution containing the base addition salt;
converting the base addition salt into the 6α,9α-difluoro-11β-hydroxy-17α-propionyloxy-16α-methyl-pregna-3-oxo-1,4-diene-17β-thiocarboxylic acid; and
isolating the 6α,9α-difluoro-11β-hydroxy-17α-propionyloxy-16α-methyl-pregna-3-oxo-1,4-diene-17β-thiocarboxylic acid, to thereby provide a purified 6α,9α-difluoro-11β-hydroxy-17α-propionyloxy-16α-methyl-pregna-3-oxo-1,4-diene-17β-thiocarboxylic acid.

According to still further features in the described preferred embodiments the fluticasone propionate has a purity that equals to or is greater than 99%, preferably equals to or is greater than 99.5%.

According to still further features in the described preferred embodiments the process further comprises, subsequent to the isolating: providing a powdered fluticasone propionate, preferably by subjecting the fluticasone propionate to spray drying.

According to still further features in the described preferred embodiments the spray drying comprises: providing a solution containing fluticasone propionate and a solvent; and spray drying the solution.

According to still further features in the described preferred embodiments the solution is substantially devoid of an additive.

According to still further features in the described preferred embodiments there are provided fluticasone propionate and powdered fluticasone propionate, prepared as described above.

According to another aspect of the present invention there is provided fluticasone propionate that has a purity that equals to or is greater than 99%, preferably that equals to or is greater than 99.5%.

According to still another aspect of the present invention there is provided a process of preparing a powdered fluticasone propionate, which comprises: providing fluticasone propionate; dissolving the fluticasone propionate in a solvent to thereby obtain a solution containing the fluticasone propionate, the solution being substantially devoid of an additive; and spray drying the solution, thereby obtaining the powdered fluticasone propionate.

According to further features in preferred embodiments of the invention described below, providing the fluticasone propionate is effected by the process described hereinabove.

According to still further features in the described preferred embodiments the powdered fluticasone propionate has at least one characteristic selected from the group consisting of: an average size that ranges from about 1 micron to about 10 micron; free flowing; a substantially spherical particles shape; and a substantial absence of an electrostatic charge.

According to still further features in the described preferred embodiments the powdered fluticasone propionate is substantially crystalline.

According to still further features in the described preferred embodiments the powdered fluticasone propionate is partially amorphous.

According to still further features in the described preferred embodiments the average particle size of the powdered fluticasone propionate ranges from about 1 micron to about 5 microns.

According to still further features in the described preferred embodiments a particle size of at least 90 percents of the particles of the powdered fluticasone propionate ranges from 1 to 5 microns.

According to still further features in the described preferred embodiments a particle size of about 50 percents of the particles of the powdered fluticasone propionate ranges from about 2 microns to about 3 microns.

According to still further features in the described preferred embodiments a particle size of about 50 percents of the particles of the powdered fluticasone propionate ranges from about 3 microns to about 5 microns.

According to still further features in the described preferred embodiments the solvent is selected from the group consisting of an alcohol, a ketone and a mixture thereof.

According to still further features in the described preferred embodiments the alcohol is selected from the group consisting of ethanol and isopropanol.

According to still further features in the described preferred embodiments the ketone is selected from the group consisting of acetone and methyl ethyl ketone.

According to still further features in the described preferred embodiments a ratio between the alcohol and the ketone in the mixture is about 1:1.

According to still further features in the described preferred embodiments the spray drying is performed at an outlet temperature greater than 60° C.

According to still further features in the described preferred embodiments the spray drying is performed at a flow of about 50 m³/hour.

According to still further features in the described preferred embodiments there is provided a powdered fluticasone propionate prepared by the process described hereinabove.

According to yet another aspect of the present invention there is provided a powdered S-fluoromethyl-6α9α-difluoro-11β-hydroxy-16α-methyl-3-oxo-17α-propionyloxyandrosta-1,4-diene-17β-carbothioate (fluticasone propionate) being characterized by at least one characteristic selected from the group consisting of: an average particle size that ranges from about 1 micron to about 10 micron, preferably from about 1 micron to about 5 microns; free flowing; a substantially spherical particles shape; and a substantial absence of an electrostatic charge.

According to further features in preferred embodiments of the invention described below, the powdered fluticasone propionate is substantially crystalline.

According to still further features in the described preferred embodiments the powdered fluticasone propionate is partially amorphous.

According to still further features in the described preferred embodiments a particle size of at least 90 percents of the particles of the powdered fluticasone propionate ranges from 1 to 5 microns.

According to still further features in the described preferred embodiments a particle size of about 50 percents of the particles of the powdered fluticasone propionate ranges from about 2 microns to about 3 microns.

According to still further features in the described preferred embodiments a particle size of about 50 percents of the particles of the powdered fluticasone propionate ranges from about 3 microns to about 5 microns.

According to still further features in the described preferred embodiments there is provided a pharmaceutical composition formulated for administration by inhalation, which comprises the powdered fluticasone propionate described herein.

According to an additional aspect of the present invention there is provided a process of purifying 6α,9α-difluoro-11β-hydroxy-17α-propionyloxy-16α-methyl-pregna-3-oxo-1,4-diene-17β-thiocarboxylic acid, which is effected essentially as described hereinabove.

The present invention successfully addresses the shortcomings of the presently known configurations by providing a novel, yet simple, process of preparing highly pure fluticasone propionate in high yield and a novel, yet simple, process of preparing a dry powder form of fluticasone propionate, which is highly suitable for use in administration be inhalation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein the term “mixture” describes a mixture that includes more than one substance and which can be in any form, for example, as a homogenous solution, a suspension, a dispersion, a biphasic system and more.

As used in this application, the singular form “a,” “an,” and “the” include plural references unless the context clearly dictates otherwise. For example, the term “an agent” includes a plurality of agents, including mixtures thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein throughout, the term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

The term “method” or “process” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 presents a micrograph of a sample of powdered fluticasone propionate micronized by a standard air-jet milling technique as presented in Reference Example 12 in the Examples section that follows, showing the irregular jaggedly shape of the particles produced by this technique;

FIG. 2 presents the dynamic light scattering measurement performed for powdered fluticasone propionate prepared according to Example 13, showing the particles size distribution as the average size of particles per percentile, denoted as D, and demonstrating the narrow and well defined distribution of the particles, with D(0.10) of 0.95 micron, D(0.20) of 1.31 micron, D(0.50) of 2.21 microns, D(0.80) of 3.43 microns, D(0.90) of 4.23 microns, D(0.95) of 4.96 microns, D(0.98) of 5.82 microns, D(0.99) of 6.37 microns and D(1.00) of 8.11 microns;

FIG. 3 presents the X-ray power diffraction spectrum measured for powdered fluticasone propionate prepared according to Example 13, showing a discrete (spiky) character typical to a powder having particles with high content of well-ordered morphology, demonstrating the crystalline morphology of the particles produced by this process of the present invention;

FIG. 4 presents a SEM micrograph of a sample of powdered fluticasone propionate as prepared according to an exemplary process according to the present embodiments (as detailed in Example 13), showing the uniform spherical shape of the particles which contribute to the free-flowing character of the resulting powder;

FIG. 5 presents the dynamic light scattering measurement performed for powdered fluticasone propionate prepared according to Example 14, showing the particles size distribution as the average size of particles per percentile, denoted as D, and demonstrating the narrow and well defined distribution of the particles, with D(0.10) of 0.96 micron, D(0.20) of 1.27 micron, D(0.50) of 2.14 microns, D(0.80) of 3.44 microns, D(0.90) of 4.36 microns, D(0.95) of 5.27 microns, D(0.98) of 6.44 microns, D(0.99) of 7.32 microns and D(1.00) of 13.54 microns;

FIG. 6 presents the dynamic light scattering measurement performed for powdered fluticasone propionate prepared according to Example 15, showing the particles size distribution as the average size of particles per percentile, denoted as D, and demonstrating the narrow and well defined distribution of the particles, with D(0.10) of 1.06 micron, D(0.20) of 1.52 micron, D(0.50) of 2.67 microns, D(0.80) of 4.51 microns, D(0.90) of 5.95 microns, D(0.95) of 7.51 microns, D(0.98) of 9.81 microns, D(0.99) of 11.67 microns and D(1.00) of 2000.00 microns;

FIG. 7 presents the dynamic light scattering measurement performed for powdered fluticasone propionate prepared according to Example 16, showing the particles size distribution as the average size of particles per percentile, denoted as D, and demonstrating the narrow and well defined distribution of the particles, with D(0.10) of 1.90 micron, D(0.20) of 2.46 microns, D(0.50) of 4.11 microns, D(0.80) of 6.70 microns, D(0.90) of 8.40 microns, D(0.95) of 9.86 microns, D(0.98) of 11.35 microns, D(0.99) of 12.10 microns and D(1.00) of 14.24 microns; and

FIG. 8 presents the X-ray power diffraction spectrum measured for powdered fluticasone propionate prepared according to Example 16, showing a relatively continuous smooth (curve) character typical to a powder having particles with amorphous morphology and low level of crystallinity, demonstrating the mostly amorphous morphology of the particles; and

### DESCRIPTION OF THE PREFERRED EMBODIMENTS

The present invention is of (i) a novel process of preparing fluticasone propionate in high yield and purity; (ii) highly pure fluticasone propionate prepared by this process; (iii) a novel process of preparing a powdered fluticasone propionate which is highly suitable for administration by inhalation; (iv) a powdered fluticasone propionate prepared by this process; and (v) pharmaceutical compositions containing the fluticasone propionate and the powdered fluticasone propionate described above. The present invention is further of a process of purifying 6α,9α-difluoro-11β-hydroxy-17α-propionyloxy-16α-methyl-pregna-3-oxo-1,4-diene-17β-thiocarboxylic acid, a key intermediate in the synthesis of fluticasone propionate.

The principles and operation of the present invention may be better understood with reference to the drawings and accompanying descriptions.

As discussed in detail hereinabove, fluticasone propionate is a highly potent agent for the treatment of inflammatory respiratory disorders such as asthma, perennial rhinitis and of topical inflammatory conditions and is marketed worldwide.

As is further discussed in detail hereinabove, the presently known processes for preparing fluticasone propionate are limited, inter alia, by poor chemical efficiency, which lead to poor yield and purity of the final product.

In a search for an improved process of preparing fluticasone propionate, the present inventors have now surprisingly found that fluticasone propionate can be efficiently prepared in the presence of water. Specifically, it was found that fluticasone propionate can be prepared by reacting 6α,9α-difluoro-11β-hydroxy-17α-propionyloxy-16α-methyl-pregna-3-oxo-1,4-diene-17β-thiocarboxylic acid (which is also referred to herein interchangeably Compound I or simply as thiocarboxylic acid) with a halofluoromethane, in the presence of water and a base, in an appropriate organic solvent. It was further found that fluticasone propionate thus prepared can be readily isolated from the reaction mixture and is obtained in higher yield and purity as compared with the prior art processes. Such a process is highly applicable for industrial scale-up and is further beneficial since it results in fluticasone propionate having a pharmaceutical purity.

As used herein and in well accepted in the art, the phrase “pharmaceutical purity” of a substance (e.g., fluticasone propionate) describes a substance that has purity characteristics that conform to drug regulations assuring that the substance meets the requirements of the act as to safety and meets the quality it is represented to possess. Typically, a substance (e.g., drug) having a pharmaceutical purity is a substance having less than 0.50% by weight total content of impurities.

Thus, according to one aspect of the present invention there is provided a process of preparing fluticasone propionate. The process, according to this aspect of the present invention is effected by providing the thiocarboxylic acid Compound I (see, Schemes 1 and 2); reacting the thiocarboxylic acid with a halofluoromethane in the presence of water and a base, to thereby obtain a reaction mixture containing fluticasone propionate; and isolating the fluticasone propionate from the reaction mixture.

The process according to this aspect of the present invention is illustrated in Scheme 2 below.

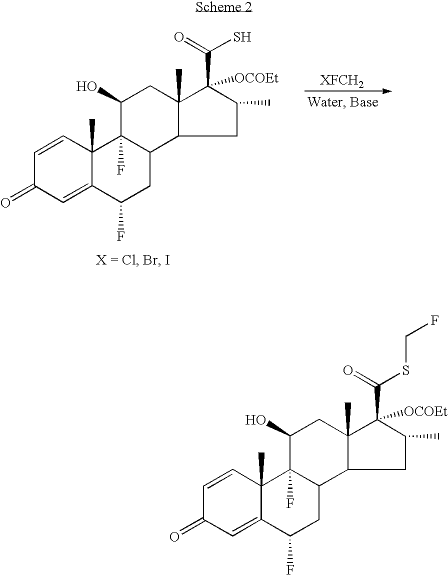

The thiocarboxylic acid (Compound I) used as the starting material in this process is known and obtainable by conventional methods known in the art, such as, for example, those described in U.S. Patent Application having the Publication No. 2004/0116396 and Israeli Patent IL 109,656.

According to a preferred embodiment of this aspect of the present invention, the thiocarboxylic acid is further purified prior to the reaction with the halofluoromethane.

The present inventors have now surprisingly found that the thiocarboxylic acid can be efficiently and easily purified by converting it to the base addition salt thereof and then re-converting the base addition salt to the free acid. As is exemplified in the Examples section that follows, such a purification process is effected in high yield.

Thus according to preferred embodiments of this aspect of the present invention and further according to another aspect of the present invention, there is provided a process of purifying 6α,9α-difluoro-11β-hydroxy-17α-propionyloxy-16α-methyl-pregna-3-oxo-1,4-diene-17β-thiocarboxylic acid (Compound I), which is generally effected by: providing the thiocarboxylic acid Compound I, using any of the methods known in the art; converting the thiocarboxylic acid into a base addition salt thereof, converting the base addition salt into the free thiocarboxylic acid; and isolating the thus obtained thiocarboxylic acid. Preferably, the base addition salt of the thiocarboxylic acid is isolated prior to its conversion to the free acid form.

Converting the thiocarboxylic acid into the base addition salt thereof can be performed by reacting the compound with a base, preferably an aqueous solution of a base, in the presence of an organic solvent.

Thus, converting the thiocarboxylic acid into the base addition salt thereof can be performed by contacting a solution containing the thiocarboxylic acid and an organic solvent with an alkaline aqueous solution, namely an aqueous solution that contains a water-soluble base.

The base used in these embodiments of the present invention is therefore preferably a water soluble base and can be an inorganic base or an organic base.

More preferably, the base is an inorganic base such as, but not limited to, sodium hydroxide, sodium carbonate, potassium carbonate, sodium bicarbonate and the like. Preferably, the base is sodium bicarbonate.

Thus, the base addition salt of the thiocarboxylic acid formed in this process can be, for example, a sodium addition salt, a potassium addition salt and the like.

The conversion of the thiocarboxylic acid to the base additional salt thereof is performed in a biphasic system comprised of an aqueous phase containing the base and an organic solvent, in which the thiocarboxylic acid (in its preliminary free form) is dissolved. Once the thiocarboxylic acid reacts with the base, the formed base addition salt is present within the aqueous phase of the biphasic system and thus can be easily separated from the reaction mixture.

Using such a biphasic system allows for an efficient isolation of the water-soluble base addition salt from the reaction mixture while water-insoluble impurities remain in the organic phase.

Converting the base addition salt of the thiocarboxylic acid in its free form is preferably performed by reacting it with an acid, preferably with an aqueous solution of an acid and more preferably with an aqueous solution of an inorganic acid such as HCl. This conversion is preferably effected while adjusting the pH of this reaction mixture to a pH lower than 4, more preferably lower than 3, and more preferably, to a pH of about 1-2.

The thus formed purified free thiocarboxylic acid is obtained as a precipitate, which is easily separated from the reaction mixture by e.g., filtration. The separated purified thiocarboxylic acid can be further dried using conventional drying procedures.

As is exemplified in the Examples section that follows, using the purification process described above, purified thiocarboxylic acid is obtained in high yield (e.g., greater than 80%) and high purity (e.g., 95.7%, as determined by HPLC).

When used as a starting material in the process of preparing fluticasone propionate presented herein, the thiocarboxylic acid may be either in a dry form or in a wet form, as is detailed hereinbelow.

Turning back to the process of preparing fluticasone propionate according to the present embodiments, once the thiocarboxylic acid is provided, it is reacted with a halofluoromethane, represented as XFCH₂, wherein X can be chloro, bromo or iodo, in Scheme 2 above.

Thus, the halofluoromethane utilized in the process according of this aspect of the present invention can be, for example, chlorofluoromethane, bromofluoromethane and iodofluoromethane, and is preferably chlorofluoromethane.

In a preferred embodiment of this aspect of the present invention, an excessive molar amount, relative to the thiocarboxylic acid, of the halofluoromethane is used in this process. Preferably, the relative amount of the halofluoromethane ranges from about 1 molequivalent to about 10 molar equivalents relative to the molar amount of the thiocarboxylic acid, more preferably from about 1 molequivalent to about 5 molequivalents and most preferably from about 2 molequivalents to about 4 molequivalents, relative to the molar amount of the thiocarboxylic acid.

As used herein throughout, the term “about” refers to ±10%.

According to a preferred embodiment of this aspect of the present invention, reacting the thiocarboxylic acid with the halofluoromethane is performed in an organic solvent.

Since the process described herein is performed in the presence of water, suitable organic solvents for use in this context of the present invention are water-miscible solvents such as, but not limited to, alcohols, ethers, nitriles, amides, ketones, sulfoxides and the like, including any combination thereof.

Non-limiting examples of water miscible organic solvents that are usable in the context of the present invention therefore include, alcohols such as, but not limited to, methanol, ethanol, propanol, isopropyl alcohol, butanol, isobutanol, t-butanol, pentanol, hexanol, cyclohexanol, and benzyl alcohol; cyclic ethers, such as, but not limited to, tetrahydrofuran, 2-methyltetrahydrofurane, pentamethylene oxide, 1,4-dioxane and the like, amides such as, but not limited to, N,N-dimethylformamide, N,N-dimethylacetamide, dimethyl sulfoxide, sulfolane, 2-pyrrolidone, N-methyl-2-pyrrolidone, 2-oxazolidone, 1,3-dimethyl-2-imidazolidinone, acetonitrile, and acetone. These solvents may be used either individually or as a combination of two or more of the foregoing.

More preferred solvents that are suitable for use in this context of the present invention include, without limitation, tetrahydrofuran, 2-methyltetrahydrofurane, acetonitrile, and any combination thereof. The presently most preferred solvent is acetonitrile.

Reacting the thiocarboxylic acid with the halofluoromethane is performed in the presence of a base.

The base can be an inorganic base or an organic base.

Non-limiting examples of inorganic bases that are suitable for use in this context of the present invention include, without limitation, metal hydroxides such as sodium hydroxide and potassium hydroxide; metal carbonates such as sodium carbonate and potassium carbonate; and metal hydrogencarbonates such as sodium hydrogencarbonate and potassium hydrogencarbonate (also referred to herein as in the art as sodium bicarbonate and potassium bicarbonate, respectively) or a combination thereof.

Non-limiting examples of organic bases that are suitable for use in this context of the present invention include, without limitation, tertiary alkylamines such as triethylamine, tributylamine and N,N-diisopropylethylamine; dialkylanilines such as N,N-dimethylaniline and N,N-diethylaniline; heterocyclic amines such as pyridine, N,N-dimethylaminopyridine and N-methylmorpholine; 1,8-diazobicyclo[5,4,0]undecene, N-benzyltrimethylammonium hydroxide and any combination thereof.

Preferably, the base is an organic base and more preferably it is a tertiary alkylamine such as triethylamine, tributylamine and N,N-diisopropylethylamine, whereby the presently most preferred base is N,N-diisopropylethylamine.

The amount of the base used in this process of the present invention preferably ranges from about 0.1 molequivalent to about 15 molequivalents relative to the thiocarboxylic acid, more preferably from about 1 molequivalent to about 10 molequivalents relative to the thiocarboxylic acid, and more preferably from about 1 molequivalent to about 5 molequivalents relative to the thiocarboxylic acid.

As discussed hereinabove, the process according to this aspect of the present invention is efficiently performed in the presence of water. As is demonstrated in the Examples section that follows, the process according to this aspect of the present invention was efficiently performed in the presence of variable amounts of water, ranging from 5 weight percentages of water relative to the weight of the thiocarboxylic acid to 200 weight percentages of water relative to the weight thiocarboxylic acid used.

The amount of the water used in this process can therefore range from about 1 weight percentage to about 200 weight percentages, preferably from about 20 weight percentages to about 100 weight percentages, and more preferably form about 40 weight percentages to about 70 weight percentages, relative to the weight of the thiocarboxylic acid.

As mentioned hereinabove, the thiocarboxylic acid utilized in this process of the present invention can be either is a dry form or is a wet form.

When utilized in a dry form, reacting the thiocarboxylic acid with the halofluoromethane in the presence of water is effected by adding the water to the reaction mixture, whereby the amount of the added water is as described hereinabove. In this case, the process according to this aspect of the present invention may further be effected by drying thiocarboxylic acid prior to reacting it with the halofluoromethane, using any of the conventional drying methods known in the art.

When utilized in a wet form, the total amount of the water used while reacting the thiocarboxylic acid with the halofluoromethane can include the water content of the thiocarboxylic used. Thus, for example, if the thiocarboxylic acid utilized in the process according to this aspect of the present invention is in a wet form, and depending on the desired total amount of water utilized in the process, as is detailed hereinabove, addition of water to the reaction mixture can be avoided. If the water content in the starting material is insufficient, water may be added directly to the reaction mixture, or may be added initially to the starting material.

Reacting the thiocarboxylic acid and the halofluoromethane, in the presence of water and a base, as described hereinabove, can be conveniently performed at room temperature. Optionally and preferably, the reaction is performed at elevated temperatures. Thus, the reaction can be carried at a temperature that ranges from room temperature and the reflux temperature of the solvent (or, in the absence of solvent, the reflux temperature of water). Preferably, the reaction is carried out at a temperature that ranges between 40° C. and 60° C.

Further, the reaction can be conveniently conducted at atmospheric pressure.

Optionally and preferably, the reaction may be carried out in a sealed high-pressure reaction vessel, under elevated pressure that may develop during the reaction.

Depending on the reaction conditions, namely, the temperature and pressure, the reaction time required to achieve high conversion of the thiocarboxylic acid to fluticasone propionate may range from 1 hour to a few days. When the reaction is conducted at elevated temperatures, as detailed hereinabove, and in a sealed reaction vessel, the reaction time preferably ranges from about 1 hour to about 10 hours, more preferably for about 5 hours.

Once the reaction is completed, the obtained fluticasone propionate can be isolated from the reaction mixture using any of the conventional techniques known in the art.

Thus, isolating the fluticasone propionate from the reaction mixture can be performed using, for example, filtration, centrifugation, extraction, evaporation, trituration, column chromatography, preparative thin-layer chromatography, preparative low, medium or high-pressure liquid chromatography or any combination of thereof, with filtration being the presently most preferred technique.

Once isolated from the reaction mixture, the fluticasone may optionally be dried, using conventionally known methods. Thus, drying the fluticasone propionate obtained by the process described herein may be carried out, for example, by increasing the temperature or reducing the pressure or a combination of both. Non-limiting examples of drying technologies or equipments usable in this context of the present invention include vacuum ovens, tray ovens, rotary ovens and fluidized bed dryers.

Using the process described herein, fluticasone propionate is obtained in high yield and purity.

As is demonstrated in the Examples section that follows, in exemplary processes performed as described herein, fluticasone propionate was obtained in a yield of about 80%.

Thus, according to an embodiment of this aspect of the present invention, using the process described herein, the fluticasone propionate is obtained in a yield greater than 60%, preferably greater than 70%, and more preferably greater than 80%, relative to the molar amount of the thiocarboxylic acid starting material (Compound I).

Such a high yield clearly demonstrates the higher efficiency of this process as compared with the presently known processes for preparing fluticasone propionate described above, in which the product was obtained in a yield of 50-60%.

As is further demonstrated in the Examples section that follows, in exemplary processes performed as described herein, the purity of the obtained fluticasone propionate was greater than 99% and even greater than 99.5% and, in some cases, even greater than 99.8%, before any purification procedure was applied. As discussed hereinabove, such purity corresponds to pharmaceutical purity.

Thus, according to preferred embodiments of this aspect of the present invention, using the process described above fluticasone propionate having a purity that is equal to or greater than 99%, preferably equal to or greater than 99.1%, more preferably equal to or greater than 99.2%, more preferably equal to or greater than 99.3%, more preferably equal to or greater than 99.4%, and even more preferably equal to or greater than 99.5%, as determined by HPLC, is obtained.

Thus, using the process described herein fluticasone propionate having a pharmaceutically acceptable purity, that is, an impurity content of no more than 0.5%, can be obtained, without the need to use laborious purification procedures.

Notwithstanding the above, once isolated and optionally dried, the obtained fluticasone propionate can be further purified, using any of the conventionally known purification procedures. Thus, the fluticasone propionate can be purified using, for example, extraction, column chromatography, preparative low-pressure liquid chromatography, preparative high-pressure liquid chromatography, re-crystallization, slurrying and any combination thereof.

In an embodiment of this aspect of the present invention, the minute amount of impurities present in the product can be removed by treating the fluticasone propionate with activated charcoal. The activated charcoal can be added to the reaction mixture, once the reaction is completed and prior to isolating the product. If desired, a filter-aid may be additionally added. According to this embodiment, when the activated charcoal is added to the reaction mixture, stirring is continued, preferably at a constant temperature and for a time period that ranges from 5 minutes to 60 minutes, preferably from 10 minutes to 30 minutes, and most preferably is about 15 minutes, and the resulting mixture is then filtered to remove the solids. The purified fluticasone propionate may be thereafter dried by any of the drying methods described hereinabove. Alternatively, the fluticasone propionate can be treated with the activated charcoal subsequent to its isolation, by dissolving the isolated product in an organic solvent and treating the resulting solution with the activated charcoal, as described hereinabove.

According to another embodiment of this aspect of the present invention, the fluticasone propionate is purified by recrystallization. Preferably, the re-crystallization is carried out from a solvent mixture of isopropanol and acetone. As is discussed in detail hereinabove, since fluticasone propionate is a potent agent for treating disorders in the respiratory tract, it is preferably formulated for administration by inhalation. As such, the fluticasone propionate is desirably used in a dry powder form which should have a specific particles size and particles size distribution, as well as other characteristics, as is discussed hereinabove and is further detailed hereinbelow.

Thus, the fluticasone propionate prepared by the process described hereinabove, can be further utilized for providing a powdered fluticasone propionate that is characterized by a desired particles size and particles size distribution.

As used herein throughout, the phrase “powdered fluticasone propionate” describes a solid substance (the fluticasone propionate) that is in a finely divided state, namely a particulate substance. In other words, this phrase describes a solid substance in the form of tiny loose particles.

Providing a powdered fluticasone propionate that is characterized by a desired particles size and particles size distribution can be effected by subjecting the fluticasone propionate obtained by the process described above to mechanical micronization.

Any of the commonly used methods of mechanical micronization can be used for that purpose, including, for example, air jet milling, spiral jet milling, and fluid bed jet milling.

Other techniques commonly utilized for obtaining particles of the desired size and size distribution can also be employed. These include super-critical fluid processing to form nanoparticles, high pressure homogenization and spray drying.

Preferably, and as is described in detail hereinbelow, the process described hereinabove further includes spray drying the fluticasone propionate obtained and more preferably, further includes spray drying the obtained fluticasone propionate using the technology described in detail hereinbelow.

Thus, according to further embodiments of this aspect of the present invention, there is provided a powdered fluticasone propionate, prepared as described hereinabove.

Using the process according to this aspect of the present invention, highly pure fluticasone propionate, optionally in a powdered form, is obtained in high yield. This process is easy to performed and can be readily, conveniently and cost-effectively scaled-up. Thus, using the process provided herein, industrial preparation of fluticasone propionate can be efficiently and advantageously performed.

Further according to the present invention there is provided highly pure fluticasone propionate. The fluticasone propionate according to this aspect of the present invention has a purity that is equal to or greater than 99%, preferably equal to or greater than 99.1%, more preferably equal to or greater than 99.2%, more preferably equal to or greater than 99.3%, more preferably equal to or is greater than 99.4%, and even more preferably equal to or greater than 99.5%, as determined by HPLC.

Further according to the present invention, there is provided a novel process of preparing a powdered fluticasone propionate that is suitable for use for administration by inhalation.

As discussed hereinabove, fluticasone propionate is mainly used in the treatment of inflammatory respiratory disorders such as asthma, and therefore its main mode of administration is by inhalation. As such, fluticasone propionate is required to comply with several precise quality standards such as maximal and minimal particular particle size, narrow particle size distribution, free flowing flow and a low tendency to agglomerate. These qualities are affected by several characteristics of the solid particles, some of which arise from the process by which the particles are obtained, and which include particle shape, surface morphology and area and the tendency to absorb moisture and accumulate static charge.

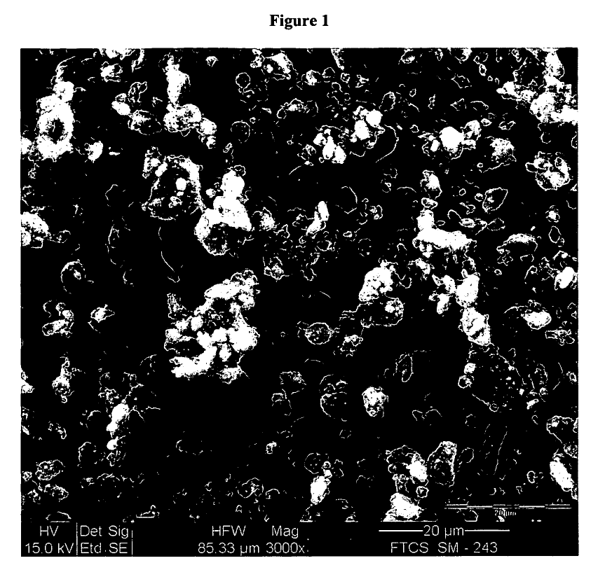

As mentioned hereinabove, the standard mechanical drug-milling and micronization techniques, such as air-jet milling, is routinely used for reduction of particle size of powders and for obtaining particle size distribution of the powders in the range of 1-10 microns. As air-jet milling equipment is available from many suppliers, the present inventors have attempted to apply this particle size reduction technique on fluticasone propionate. These attempts, presented in Example 12 in the Example section that follows, gave unsatisfactory results. The overall time-consuming process resulted in a powder which contained either too many oversized particles or too many undersized particles, and further tended to accumulate a considerable amount of static charge and generally tended to agglomerate. The present inventors have also recorded a significant reduction in the crystallinity of the final particles and an irregular shape of the particles, as is shown in the SEM micrograph presented in FIG. 1. Therefore it was concluded that mechanical air-jet milling techniques are not suitable for fluticasone propionate as these processes typically result in the disadvantageous irregular and jaggedly shaped particles having an amorphous surface, while failing to achieve the required narrow particle size distribution of 1-10 microns. These undesired powder characteristics might have a negative effect on the therapeutic activity of the drug in the respiratory tract.

In a search for an improved process for obtaining pharmaceutically acceptable dry powder form of fluticasone propionate which is suitable for administration by inhalation, the present inventors have turned to the well established technique known as spray drying.

Spray drying is the most widely used industrial process involving particle formation and drying. It is highly suited for the continuous production of dry solids in either powder, granulate or agglomerate form from liquid feedstocks such as solutions, emulsions and pumpable suspensions. Spray drying involves the atomization of a liquid feedstock into a spray of droplets and contacting the droplets with hot air in a drying chamber until all solvent is evaporated from the droplets. The sprays are produced by either rotary (wheel) or nozzle atomizers. The evaporation of the solvent from the droplets and formation of dry particles proceed under controlled temperature and airflow conditions, and the resulting powder is discharged continuously from the drying chamber.

The most crucial parts of the spray drying process are the atomization of the droplets and the evaporation of the solvent. Both processes can be affected by the type of the liquid carrier used, stemming from physical and/or mechanical properties of the carrier such as viscosity and rheological characteristics, boiling point, gas content, hygroscopicity and the like, as well as the solubility of the drug in a particular liquid carrier. The carrier therefore determines the shape and size of the droplets, the size distribution and the amount of lingering carrier residual.

The carrier used in a spray drying process aimed at producing well-defined powders is desirably selected capable of dissolving relatively high drug content (high drug solubility in the carrier), having adequate physical and/or mechanical properties so as to allow the formation of small and uniform spherical droplets and high evaporation rate at moderate to low temperatures.

As mentioned above, attempts to apply a conventional spray drying technique to drugs such as fluticasone propionate have met limited success. Complicated related techniques which utilized either multi-phasic conditions, multi-solvent systems and other cost- and time-consuming processes have been used. Alternatively, conventional spray drying technique was used while disadvantageously adding various additives to the liquid feedstock in order to achieve the required solution properties which lead to the desirable powders properties of the drug.

In a search for an industrially applicable and efficient process for preparing powdered fluticasone propionate that is suitable for administration by inhalation, the present inventors have now surprisingly found that a powdered fluticasone propionate characterized by the desired properties, as is detailed herein, can be achieved using the conventional spray drying technique while circumventing the need to use additives or any other complicated procedures.

Hence, according to further aspects of the present invention, there are provided a powdered S-fluoromethyl-6α,9α-difluoro-11β-hydroxy-16α-methyl-3-oxo-17α-propionyloxyandrosta-1,4-diene-17β-carbothioate (fluticasone propionate), as this phrase is defined hereinabove, suitable for administration by inhalation, and a process for obtaining same.

The process, according to these aspects of the present invention, is effected by:
providing fluticasone propionate; dissolving the fluticasone propionate in a solvent to thereby obtain a solution containing the fluticasone propionate, whereby the solution is substantially devoid of an additive; and spray drying the solution, using a conventional spray drying apparatus and technique, as described hereinabove.

According to an embodiment of this aspect of the present invention, the process is effected by providing a solution of fluticasone propionate whereby the solution is consisting essentially of fluticasone propionate and solvent and spray drying this solution.

The fluticasone propionate used in the process described herein can be obtained commercially or prepared according to any of the known procedures described in the art and delineated supra. Optionally and preferably, the fluticasone propionate can be prepared using the highly efficient process described hereinabove.

Thus, according to a preferred embodiment of the present invention, the fluticasone propionate used in the process according to this aspect of the present invention is prepared by the process described hereinabove. Highly pure fluticasone propionate, having a purity as high as about 99.8%, is therefore utilized herein.

As mentioned above, a critical component of the spray drying process, which is crucial for obtaining the desired final product is the liquid carrier in which the drug is dissolved prior to the spray drying stage. This liquid carrier, also referred to herein and in the art as “feedstock”, is referred to herein interchangeably as a “solution” which includes the fluticasone propionate and a solvent.

The present inventors have now uncovered that by selecting an appropriate solvent in which the fluticasone propionate is dissolved prior to the spray drying process, a powder having the desired characteristics is obtained.

Appropriate solvents that are suitable for use in this context of the present invention thus include, without limitation, water, alcohols, ketones, acetates, ethers, nitrites and aprotic polar solvents.

More preferred solvents that are suitable for use in this context of the present invention include, low alcohols (having 2-8 carbon atoms) such as, but not limited to, methanol, ethanol, n-propanol, isopropanol, n-butanol, and isobutanol; low ketones (having 2-8 carbon atoms) such as, but not limited to, acetone, methyl ethyl ketone, diethyl ketone, methyl propyl ketone and methyl isobutyl ketone; low acetates (having 2-8 carbon atoms) such as, but not limited to, ethyl acetate, n-propyl acetate, isopropyl acetate, n-butyl acetate and isobutyl acetate; low ethers (having 2-8 carbon atoms) such as, but not limited to, diethyl ether, diisopropyl ether, methyl tert-butyl ether and tetrahydrofurane; low nitrites such as, but not limited to, acetonitrile and propionitrile; dimethylformamide (DMF), and any mixture thereof.

The presently most preferred solvents that are suitable for use in this context of the present invention are low alcohols such as ethyl alcohol and isopropanol (also referred to herein as isopropyl alcohol), low ketones such as acetone and methyl ethyl ketone, and any mixture thereof.

When a mixture of two solvents is used, the ratio between the solvents can range from 1:99 to 99:1, and preferably ranges from 1:10 to 10:1, more preferably from 1:5 to 5:1, more preferably from 1:2 to 2:1 and most preferably is 1:1.

The solution obtained by dissolving the fluticasone propionate in the solvent described hereinabove is substantially devoid of an additive.

As used herein, the term “additive” describes a substance that is typically chemically inert and is added to another substance or a mixture of substances, before, during or subsequent to applying a certain process on the substance or the mixture, and which is aimed at providing the resulting product with certain physical and/or mechanical characteristics. Additives are typically used, for example, in spray drying processes, crystallization procedures, milling processes and the like, and are aimed, for example, at modulating the solubility of a substance during the process, enhancing the stability (mechanical or chemical) of a resulting product, modulating the viscosity and/or other mechanical parameters of a mixture, affecting the morphology of the surface and crystal lattice of the resulting solids and the like. Exemplary additives that are commonly used in spray drying processes include, without limitation, lactose, polyvinyls such as PVA and polyvinylpyrrolidone (PVP), phospholipid surfactants, nonionic detergents, nonionic block copolymers, ionic surfactants and biocompatible fluorinated.

By “substantially devoid of an additive” it is meant that the solution contains less than 1% of the additive, preferably less than 0.5% of the additive and more preferably less than 0.1% of the additive, by weight. More preferably, the solution is completely devoid of an additive.

Circumventing the need to use additives, as taught herein, is beneficial since the product obtained by the process described herein is highly pure, the need to remove the additives subsequent to obtaining the powdered product is circumvented and the use of excessive chemicals is avoided, hence formulating cost is reduced.

Dissolving the fluticasone propionate in the solvent is typically performed, according to preferred embodiments of the present invention either at room temperature or at elevated temperatures and is thus preferably performed at a temperature that ranges from ambient temperature to the reflux temperature of the selected solvent. Dissolving the fluticasone propionate in the solvent is therefore typically carried out at a temperatures that ranges from 20° C. to 90° C., depending on the solvent used.

The concentration of the fluticasone propionate in the resulting solution preferably ranges from about 1 gram of the fluticasone propionate per 100 ml of the solution to about 50 grams of the fluticasone propionate to 100 ml of solution, more preferably from about 1 gram of the fluticasone propionate per 100 ml of the solution to about 10 grams of the fluticasone propionate to 100 ml of solution, and more preferably is about 5 grams of the fluticasone propionate to 100 ml of solution.

The obtained hot solution is then charged on a conventional spray drying apparatus, as exemplified in the Examples section that follows, and subjected to a conventional spray drying procedure, as is further exemplified is the Examples section that follows.

According to a preferred embodiment of the process according to this aspect of the present invention, the spray drying is carried out at the following conditions: The outlet temperature of the spray dryer is preferably set on a temperature greater than 60° C., and in some cases greater than 80° C. and even greater than 85° C. The flow rate is set on between 20 m³/hour and 100 m³/hour, preferably between 40 m³/hour and 60 m³/hour and more preferably is set on 50 m³/hour.

Other preferred parameters of the spray drying processes include an inlet temperature greater that ranges from 100° C. and 200° C., preferably from 140° C. to 180° C., and more preferably is 160° C.

As is exemplified in the Examples section that follows, the process described above, a powdered fluticasone propionate that has one or more of the following characteristics is obtained:
an average particles size that ranges from about 1 micron to about 10 micron and preferably from about 1 micron to about 5 microns;
a particle size distribution in which more than 90% of the particles have a size of from about 1 micron to about 5 microns;
free flowing;
a substantially spherical particles shape; and
a substantial absence of an electrostatic charge.

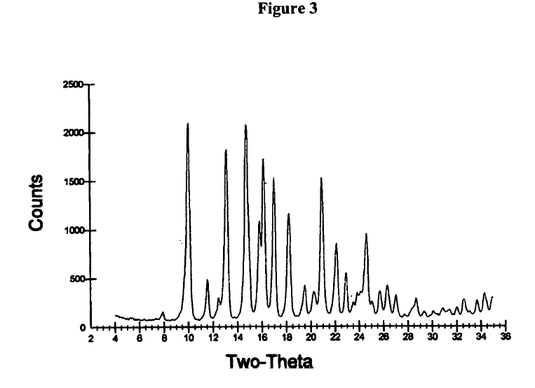

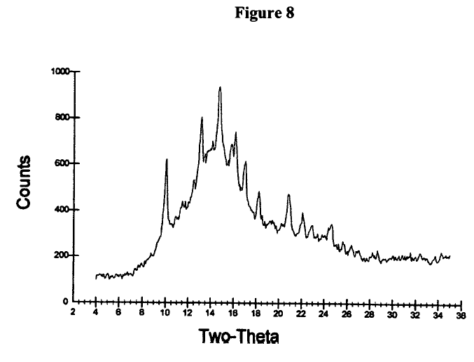

In addition, and depending on the solvent used, the resulting powdered fluticasone propionate can be either crystalline or partially amorphous. Thus, for example, when a mixture of a low alcohol and a low ketone, as described hereinabove, is used, a substantially crystalline powder of fluticasone propionate is obtained, as is shown, for example, in FIG. 3. When a solvent including only acetone was used, partially amorphous powder of fluticasone propionate was obtained, as is shown, for example, in FIG. 8.

Thus, the process according to this aspect of the present invention, is further beneficial as it enables, by appropriately selecting the solvent used, to control the physical characteristics of the obtained powdered fluticasone propionate.

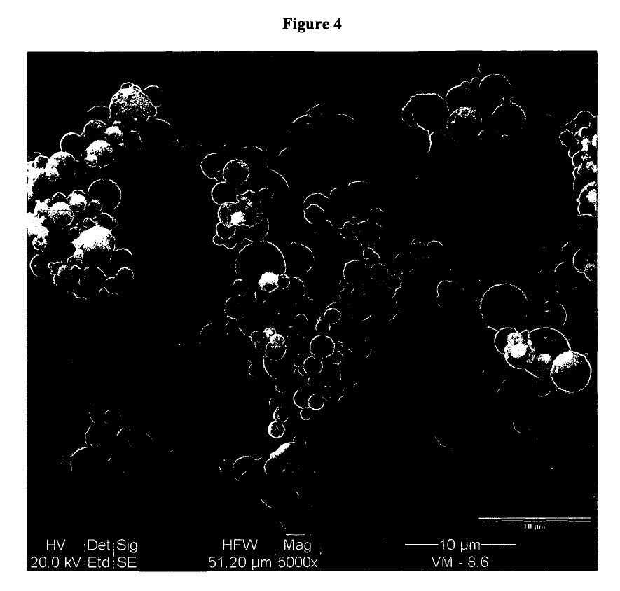

As is exemplified in the SEM micrograph presented FIG. 4, exemplary particles obtained by the process described herein have a spherical and a substantially ordered (crystalline) morphology. Further, it was recorded (data not shown) that particles devoid of electrostatic charge, namely uncharged particles, are obtained. As discussed hereinabove, such properties attribute to the free flowing of the powder and substantially reduce its tendency to agglomerate.

Indeed, it was observed that powdered fluticasone propionate prepared by the process according to this aspect of the present invention is characterized as a free flowing powder.

The particle shape obtained by the process described herein is mostly spherical, an attribute which contributes to the dry powder flow and the overall desirable properties of the final product.

As presented in the Examples section that follows, the powdered fluticasone propionate obtained by the process, according to this aspect the present invention, is desirably characterized by an average particle size that ranges from about 1 micron to about 10 micron, and more preferably from about 1 micron to about 5 micron.

Moreover, as is further presented in the Examples section that follows, the powdered fluticasone propionate obtained by the process according to this aspect the present invention is further desirably characterized by a particle size distribution in which at least 90% and preferably at least 95% of the particles in the powder have a size in the 1 to 10 micron range. The powdered fluticasone propionate obtained by the process, according to this aspect the present invention, is further desirably characterized by a particle size distribution in which at least 85% and preferably at least 90% of the particles of the powder have a size in the 1 to 5 micron range.

In addition, no more than 10% and preferably no more than 5% of the particles in the powdered fluticasone propionate obtained by the process, according to this aspect the present invention, have a size larger than 5 microns and/or smaller than 1 micron.

Furthermore, as is further demonstrated in the Examples section that follows, in certain embodiments of the process according to the present invention, about 50% of the particles of the obtained powdered fluticasone propionate have a particle size which ranges from about 2 microns to about 3 microns. Such a particles size distribution is typically obtained when a mixture of a low alcohol and a low ketone is used and the powder has a crystalline morphology.

In other embodiments of the process according to the present invention, about 50% of the particles of the obtained powdered fluticasone propionate have a particle size which ranges from about 3 microns to about 5 microns. Such a particles size distribution is typically obtained when a low ketone is used and the powder has a partially amorphous morphology.

The effect of the solvent used on the particles size and size distribution further emphasize the ability to control the physical characteristics of the obtained powder, while using the process described herein.

As one of the important traits of a powdered form of a pharmaceutically active agent designed for delivery to the respiratory tract is a narrow particle size distribution, the process according to this aspect of the present invention successfully achieves this attribute, as is detailed hereinabove and is further demonstrated in the Examples section that follows.

The process described herein is highly efficient, simple to perform and can be readily scaled-up for industrial manufacture of a powdered fluticasone propionate. The powdered fluticasone propionate obtained by this process is, as is detailed hereinabove, characterized by properties which renders it highly suitable for use for administration by inhalation.

Thus, according to an additional aspect of the present invention there is provided a powdered fluticasone propionate having one or more of the following characteristics: an average particles size that ranges from about 1 micron to about 10 micron and preferably from about 1 micron to about 5 microns; a particle size distribution in which more than 90% of the particles have a size of from about 1 micron to about 5 microns; free flowing; a substantially spherical particles shape; and a substantial absence of an electrostatic charge, as is detailed hereinabove, and which can be either crystalline or partially amorphous, as is detailed hereinabove.

As discussed in detail hereinabove, the powdered fluticasone propionate described herein can be beneficially incorporated in a pharmaceutical composition and particularly is a pharmaceutical composition that is formulated fro admisnitration by inhalation.

Thus, according to still an additional aspect of the present invention there are provided pharmaceutical compositions that are formulated for administration by inhalation. Each of these pharmaceutical computations includes, as an active ingredient, any of the powdered fluticasone propionate described in this and other aspects of the present invention.

Preferably, such pharmaceutical compositions are identified for use in the treatment of a disorder in the respiratory tract, and particularly of a respiratory inflammation associated with e.g., asthma, and perennial rhinitis.

Each of the powdered fluticasone propionate described herein, either per se or as a part of the pharmaceutical compositions described herein, can be further incorporated in a medical device designed for delivering an active agent to the respiratory tract.

As is described hereinabove, exemplary devices that are designed for delivering an active agent to the respiratory tract includes inhalers, whereby the most commonly used inhalers are a metered-dose inhaler (MDI) and a dry powder inhaler (DPI).

Thus, according to an embodiment of the present invention there is provided a metered-dose inhaler that comprises a powdered fluticasone propionate as described herein.

According to another embodiment of the present invention there is provided a dry powder inhaler that comprises a powdered fluticasone propionate as described herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Experimental Methods:

HPLC analyses were performed on a Hewlett Packard's HP 1050 HPLC apparatus, equipped with a Spherisorb ODSI column (5p, 250×4.6 mm) and a UV detector operated at 240 nm. Analyses conditions were as follows: Mobile phase: 42% 0.02 M ammonium dihydrogen phosphate buffer, pH=3.5, 14% acetonitrile and 44% methanol; Flow rate=1.5 ml/minute; Injection volume: 20 μl; Oven Temp.=25° C.; Run Time=twice the retention time of fluticasone propionate.

The dihydrogen phosphate buffer was prepared as follows: about 2.3 grams of ammonium dihydrogen phosphate were placed in a 1000 ml volumetric flask, dissolved in water and volume was completed to 1000 ml with water. pH was adjusted to 3.5 with phosphoric acid.

X-ray diffraction data were acquired using a PHILIPS X-ray diffractometer model PW1050-70 set at 40 kV, 28 mA, using 1.54178 Å (K_{α}), diversion slit of 1°, receiving slit of 0.2 mm, scattering slit of 1° and a graphite monochromator.

Spray drying was performed on a mini spray dryer by Buchi model B-190, with heater set to 1.8 KW to afford a temperature range of 40-220° C., and an evaporation rate of approximately 1500 ml per hour.

Particle size was measured on a dynamic light scattering device by Malvern model Mastersizer 2000, using a measuring range of 0.02-2000 μm, accuracy level of 1% at the median, helium neon laser as a red light source, and solid state light source as a blue light source.

Particle size distributions are presented as the average particle size of specified percentiles, including the flanking 0.1 and 0.9 quantiles and the median 0.5 quantile of the overall particle size distribution versus volume curve.

Milling was carried out using ISOPAK SuperJet Mill, produced by APTM, equipped with grinding chamber of 8″ diameter having capacity of about 1000 grams per hour.

### Example 1

### Preparation of highly pure fluticasone propionate

Ten (10) grams of (6S,9R,10S,11S,13S,16R,17R)-6,9-difluoro-11-hydroxy-10,13,16-trimethyl-3-oxo-17-(propionyloxy)-6,7,8,9,10,11,12,13,14,15,16,17-dodecahydro-3H-cyclopenta[a]phenanthrene-17-carbothioic S-acid (Compound I) and 50 ml acetonitrile were placed in a 200 ml glass autoclave vessel and the resulting mixture was stirred. Six (6) ml of water and 5.6 ml of diisopropylethylamine were added to the vessel while stirring. The reaction mixture was heated to a temperature of about 50° C. and was stirred for 15 minutes, to afford a clear yellow solution.

3.2 grams of chlorofluoromethane were bubbled through a dip-pipe into the stirred mixture and the autoclave was sealed. A pressure of about 0.5 bar was developed while the mixture was heated for a period of 5 hours. During the reaction, a suspension was produced. Thereafter the vent of the autoclave was opened and the mixture was cooled to room temperature. The mixture was stirred at room temperature for additional 16 hours. The obtained suspension was cooled to 5° C. and was stirred for 1 hour. The obtained solid product was filtered, washed with cold acetonitrile and dried at 60° C. to afford 8.4 grams of fluticasone propionate (80% yield), having a purity of 99.8% as determined by HPLC.

Recrystallization was performed by dissolving 6.4 grams of fluticasone propionate in a solvent mixture of 130 ml acetone and 60 ml isopropanol while mixing. The solution was heated to 30-35° C., about 0.6 gram of activated carbon was added hereto and mixing was continued for about half an hour. The solution was then concentrated under reduced pressure, followed by cooling the resulting mixture to 10-15° C. for about an hour. The obtained precipitate was filtered off, washed twice with isopropanol and dried for 8 hours under vacuum to obtain 5.7 grams of recrystallized fluticasone propionate (89% yield).

### Examples 2-10

Using the exemplary process described in Example 1 above, a series of ents with varying amounts of water were performed. The results are ized in Table 1 below:

TABLE 1

| | Weight percents of | | | |
|---|---|---|---|---|
| | added water (relative to the | | | Purity after |
| Example | weight of Compound I) | Yield | Purity | Crystallization |
| 2 | 0% | 61 | 99.52 | 99.52 |
| 3 | 5% | 55 | 99.56 | 99.62 |
| 4 | 10% | 62 | 99.66 | 99.63 |
| 5 | 20% | 80 | 99.80 | 99.76 |
| 6 | 40% | 75 | 99.50 | 99.58 |
| 7 | 60% | 80 | 99.36 | 99.57 |
| 8 | 80% | 78 | 99.26 | 99.61 |
| 9 | 100% | 81 | 99.50 | 99.56 |
| 10 | 200% | 85 | 99.19 | 99.53 |

### Example 11

### Purification of Compound I

Fifty (50) ml of a 5% sodium carbonate solution and 25 ml of ethyl acetate were placed in a 250 ml reaction vessel equipped with a magnetic stirrer. Five grams of Compound I were added to the vessel and the reaction mixture was stirred at room temperature for a period of about 45 minutes, to afford a clear biphasic solution. The two layers were then separated by means of a separating funnel and the aqueous layer was cooled to about 12° C. A solution of 4.1 ml of 32% HCl and 4.1 ml of water was added stepwise and the pH was adjusted to 1-2. The resulting suspension was stirred for 30 minutes at about 12° C. The resulting solid was filtered, washed with cold water until the washing solution showed a neutral pH, and dried at 60° C. in an air oven to yield 4.0 grams of Compound I (80% yield) having a purity of 95.7% as determined by HPLC.

### Example 12

1000 grams of pure fluticasone propionate, prepared as described hereinabove, were loaded onto an air-jet milling device equipped with grinding chamber of 8″ diameter. The device was operated for about 60 minutes.

FIG. 1 presents a SEM micrograph of the particles obtained by this milling procedure and clearly shows the irregular jaggedly shaped particles obtained. It is assumed that such a particles shape was obtained as a result of the mechanical force applied against the crystal lattice forces in this technique. Application of such a mechanical force further leads to a reduction of the substance crystallinity, and to an increase in activated surface and surface area, as compared with the non-milled crystalline substance. In addition, the obtained particles had unsatisfactory size distribution. Thus, it was found that the obtained product contained either too many oversized particles, too many undersized particles or both (data not shown). In addition, it was observed that the product tended to accumulate a considerable amount of electrostatic charge and therefore exhibited a high tendency to agglomerate.

### Example 13

Five grams of pure fluticasone propionate, prepared as described hereinabove, were dissolved in 100 ml of a 1:1 mixture of isopropanol and methyl ethyl ketone at 60° C., and kept at that temperature until complete dissolution was observed. The hot solution was then transferred into the spray dryer at an outlet temperature of 87° C. and flow of 50 m³/h.

The resulting solid was a free flowing crystalline powder which did not exhibit a tendency to agglomerate.

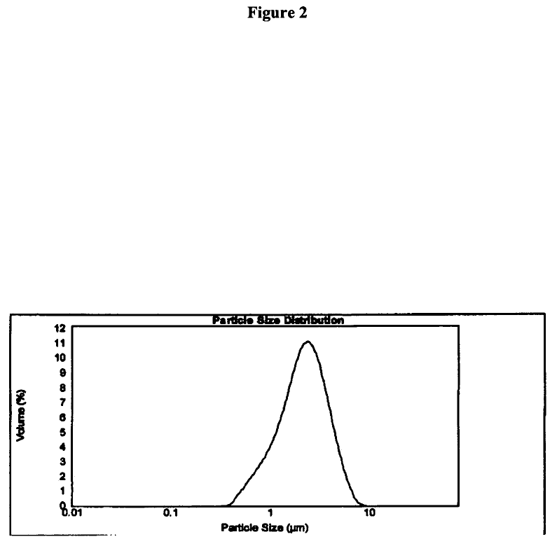

FIG. 2 presents the results of a light scattering device measurement of the particle size distribution. As can be seen in FIG. 2, the particle size distribution was found to be 0.95μ in the 0.1 quantile; 2.21μ in the 0.5 quantile; 4.23μ in the 0.9 quantile; and 4.96μ in the 0.95 quantile.

FIG. 3 presents the X-ray power diffraction pattern of the resulting powder. As is clearly shown in FIG. 3, the XRD pattern of the obtained powder is spiky and thus indicates a crystalline morphology of the final product.

FIG. 4 presents a SEM micrograph of the obtained powder. As can be seen in FIG. 4, the particles are of regular spherical shape and have smooth surface. Comparing the SEM micrograph presented in FIG. 4 (showing the fluticasone powder prepared according to the present embodiments) to that presented in FIG. 1 (showing the fluticasone powder prepared by air jet milling) clearly shows the superior shape and morphology of the particles obtained by the process of the present invention.

In addition, the product obtained in this process did not exhibit any static electricity and further did not exhibit any tendency to agglomerate.

### Example 14

Five grams of pure fluticasone propionate were dissolved in 100 ml of a 1:1 mixture of isopropanol and acetone at 60° C., and kept at this temperature until complete dissolution was observed. The hot solution was then transferred into the spray dryer at an outlet temperature of 64° C. and flow of 50 m³/h. 64° C.

The resulting solid was obtained as a free flowing crystalline powder which did not exhibit a tendency to agglomerate.

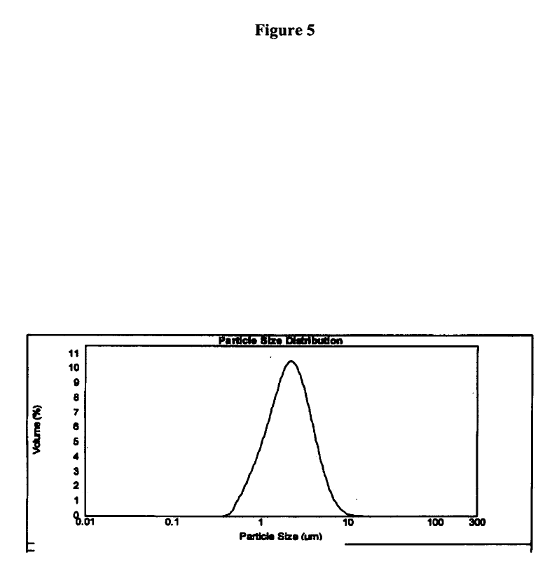

FIG. 5 presents the results of a light scattering device measurement of the particle size distribution. As can be seen in FIG. 4, the particle size distribution was found to be 0.96μ in the 0.1 quantile; 2.14μ in the 0.5 quantile; 4.36μ in the 0.9 quantile; and 5.27μ in the 0.95 quantile.

### Example 15

Five grams of pure fluticasone propionate were dissolved in 100 ml of a 1:1 mixture of ethanol and methyl ethyl ketone at 60° C., and kept at this temperature until complete dissolution was observed. The hot solution was then transferred into the spray dryer at an outlet temperature of 87° C. and flow of 50 m³/h. 64° C.

The resulting solid was obtained as a free flowing crystalline powder which did not exhibit a tendency to agglomerate.

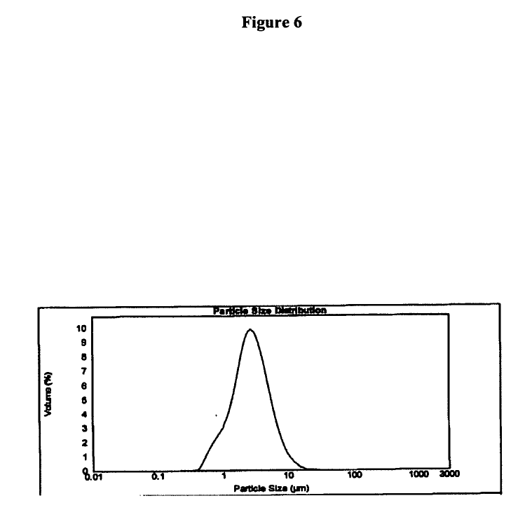

FIG. 6 presents the results of a light scattering device measurement of the particle size distribution. As can be seen in FIG. 6, the particle size distribution was found to be 1.06μ in the 0.1 quantile; 2.67μ in the 0.5 quantile; and 5.95μ in the 0.9 quantile.

### Example 16

Five grams of pure fluticasone propionate were dissolved in 100 ml of acetone at 50° C., and kept at this temperature until complete dissolution was observed. The hot solution was then transferred into the spray dryer at an outlet temperature of 80° C. and flow of 50 m³/h.

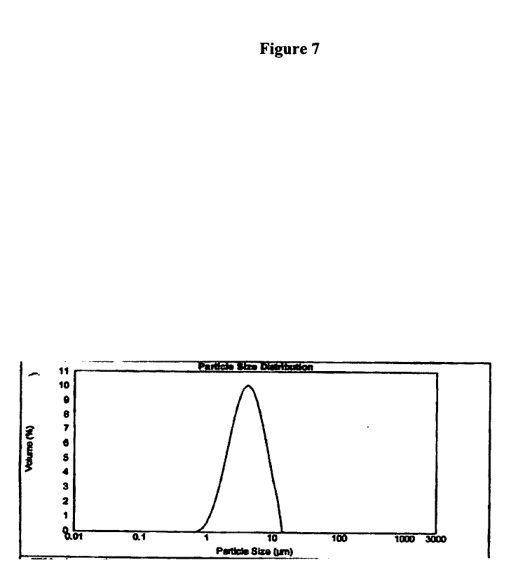

FIG. 7 presents the results of a light scattering device measurement of the particle size distribution. As can be seen in FIG. 7, the particle size distribution was found to be 1.90μ in the 0.1 quantile; 4.11μ in the 0.5 quantile; and 8.40μ in the 0.9 quantile.

FIG. 8 presents the X-ray power diffraction pattern of the resulting powder. As can be seen in FIG. 8, the XRD pattern of the powder is a relatively continuous and smooth curve, indicating a low level of a crystalline morphology of the final product.

Thus, the resulting solid was obtained as a partially amorphous powder having a particle size of less than 10 microns.

The various parameters used in the exemplary processes according to the present invention described in Examples 13-16 hereinabove and their effect on the characteristics of the resulting powder are summarized in Table 2 below.

TABLE 2

| | Solvent or solvent | | |
|---|---|---|---|
| Example | mixture | Process parameters | Powder characteristics |
| 13 | 1:1 mixture of | Dissolving at 60° C. and | Free flowing crystalline powder |
| | isopropanol and | maintaining the | that does not tend to agglomerate. |
| | methyl ethyl ketone | temperature until complete | Particle size median: 2.21 microns |
| | | dissolution. Hot solution | Particle size distribution: |
| | | was fed into the spray | D(v, 0.1) = 0.95μ; D(v, 0.5) = 2.21μ; |
| | | dryer at outlet temperature | D(v, 0.9) = 4.23μ; D(v, 0.95) = 4.96μ. |
| | | of 87° C. and flow of 50 m³/h | |
| 14 | 1:1 mixture of | Dissolving at 60° C. and | Free flowing crystalline powder |
| | isopropanol and | maintaining the | that does not tend to agglomerate. |
| | acetone | temperature until complete | Particle size median: 2.14 microns; |
| | | dissolution. Hot solution | Particle size distribution: |
| | | was fed into the spray | D(v, 0.1) = 0.96μ; D(v, 0.5) = 2.14μ; |
| | | dryer at outlet temperature | D(v, 0.9) = 4.36μ; D(v, 0.95) = 5.27μ |
| | | of 64° C. and flow of 50 m³/h | |
| 15 | 1:1 mixture of | Dissolving at 60° C. and | Free flowing crystalline powder |
| | ethanol and methyl | maintaining the | that does not tend to agglomerate. |
| | ethyl ketone | temperature until complete | Particle size median 2.67 microns; |
| | | dissolution. Hot solution | Particle size distribution: |
| | | was fed into the spray | D(v, 0.1) = 1.06μ; D(v, 0.5) = 2.67μ; |
| | | dryer at outlet temperature | D(v, 0.9) = 5.95μ. |
| | | of 87° C. and flow of 50 m³/h | |
| 16 | Acetone | Dissolving at 50° C. and | Partially amorphous powder. |
| | | maintaining the | Particle size median 4.10 microns; |
| | | temperature until complete | Particle size distribution: |
| | | dissolution. Hot solution | D(v, 0.1) = 1.90μ; D(v, 0.5) = 4.10μ; |
| | | was fed into the spray | D(v, 0.9) = 8.39μ D(v, 0.95) = 9.86μ. |
| | | dryer at outlet temperature | |
| | | of 80° C. and flow of 50 m³/h | |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A process of preparing S-fluoromethyl-6α,9α-difluoro-11β-hydroxy-16α-methyl-3-oxo-17α-propionyloxyandrosta-1,4-diene-17β-carbothioate (fluticasone propionate), the process comprising:
providing 6α,9α-difluoro-11 β-hydroxy-17α-propionyloxy-16α-methyl-pregna-3-oxo-1,4-diene-17β-thiocarboxylic acid;
reacting said 6α,9α-difluoro-11β-hydroxy-17α-propionyloxy-16α-methyl-pregna-3-oxo-1,4-diene-17β-thiocarboxylic acid with a halofluoromethane in the presence of an organic solvent, water and a base, to thereby obtain a reaction mixture containing fluticasone propionate; and
isolating the fluticasone propionate from said reaction mixture, thereby obtaining the fluticasone propionate.

2. The process of claim 1, wherein said halofluoromethane is selected from the group consisting of chlorofluoromethane, bromofluoromethane and iodofluoromethane.

3. The process of claim 1, wherein said organic solvent is selected from the group consisting of tetrahydrofuran, 2-methyltetrahydrofurane, acetonitrile and any mixture thereof.

4. The process of claim 1, wherein said base is a tertiary alkylamine.

5. The process of claim 1, wherein an amount of said water ranges from about 1 weight percent to about 200 weight percents of the weight of said 6α,9α-difluoro-11β-hydroxy-17β-propionyloxy-16α-methyl-pregna-3-oxo-1,4-diene-17β-thiocarboxylic acid.

6. The process of claim 5, wherein an amount of said water ranges from about 40 weight percents to about 70 weight percents of the weight of said 6α,9α-difluoro-11β-hydroxy-17α-propionyloxy-16α-methyl-pregna-3-oxo-1,4-diene-17β-thiocarboxylic acid.

7. The process of claim 1, further comprising, prior to said reacting, purifying said 6α,9α-difluoro-11β-hydroxy-17α-propionyloxy-16α-methyl-pregna-3-oxo-1,4-diene-17β-thiocarboxylic acid.

8. The process of claim 7, wherein said purifying comprises:
providing a solution of said 6α,9α-difluoro-11β-hydroxy-17α-propionyloxy-16α-methyl-pregna-3-oxo-1,4-diene-17β-thiocarboxylic acid and an organic solvent;
contacting said solution with an aqueous solution containing a base, to thereby provide an aqueous solution containing a base addition salt of said 6α,9α-difluoro-11β-hydroxy-17α-propionyloxy-16α-methyl-pregna-3-oxo-1,4-diene-17β-thiocarboxylic acid;
isolating said aqueous solution containing said base addition salt;
converting said base addition salt into said 6α,9α-difluoro-11β-hydroxy-17α-propionyloxy-16α-methyl-pregna-3-oxo-1,4-diene-17β-thiocarboxylic acid; and
isolating said 6α,9α-difluoro-11β-hydroxy-17α-propionyloxy-16α-methyl-pregna-3-oxo-1,4-diene-17β-thiocarboxylic acid, to thereby provide a purified 6α,9α-difluoro-11β-hydroxy-17α-propionyloxy-16α-methyl-pregna-3-oxo-1,4-diene-17β-thiocarboxylic acid.

9. The process of claim 1, wherein said fluticasone propionate has a purity that equals to or is greater than 99%.

10. The process of claim 9, wherein said fluticasone propionate has a purity that equals to or is greater than 99.5%.

11. The process of claim 1, further comprising, subsequent to said isolating:
providing a powdered fluticasone propionate.

12. The process of claim 11, wherein providing said powdered fluticasone propionate comprises:
subjecting said fluticasone propionate to spray drying.

13. The process of claim 12, wherein said spray drying comprises:
providing a solution containing fluticasone propionate and a solvent; and
spray drying said solution.

14. The process of claim 13, wherein said solution is substantially devoid of an additive.

15. Fluticasone propionate prepared by the process of claim 1.

16. Powdered fluticasone propionate prepared by the process of claim 13.

17. Fluticasone propionate having a purity that equals to or is greater than 99%.

18. The fluticasone propionate of claim 17, having a purity that equals to or is greater than 99.5%.

19. A process of preparing a powdered fluticasone propionate, the process comprising:
providing fluticasone propionate;
dissolving said fluticasone propionate in a solvent to thereby obtain a solution containing said fluticasone propionate, said solution being substantially devoid of an additive; and
spray drying said solution, thereby obtaining the powdered fluticasone propionate.

20. The process of claim 19, wherein providing said fluticasone propionate comprises:
providing 6α,9α-difluoro-11β-hydroxy-17α-propionyloxy-16α-methyl-pregna-3-oxo-1,4-diene-17β-thiocarboxylic acid;
reacting said 6α,9α-difluoro-11β-hydroxy-17α-propionyloxy-16α-methyl-pregna-3-oxo-1,4-diene-17β-thiocarboxylic acid with a halofluoromethane in the presence of an organic solvent, water and a base, to thereby obtain a reaction mixture containing fluticasone propionate; and
isolating the fluticasone propionate from said reaction mixture, thereby obtaining the fluticasone propionate.

21. The process of claim 19, wherein the powdered fluticasone propionate has at least one characteristic selected from the group consisting of:
an average size that ranges from about 1 micron to about 10 micron;
free flowing;
a substantially spherical particles shape; and
a substantial absence of an electrostatic charge.

22. The process of claim 21, wherein the powdered fluticasone propionate is substantially crystalline.

23. The process of claim 21, wherein the powdered fluticasone propionate is partially amorphous.

24. The process of claim 21, wherein said average particle size ranges from about 1 micron to about 5 microns.

25. The process of claim 24, wherein a particle size of at least 90 percents of the particles of the powdered fluticasone propionate ranges from 1 to 5 microns.

26. The process of claim 22, wherein a particle size of about 50 percents of the particles of the powdered fluticasone propionate ranges from about 2 microns to about 3 microns.

27. The process of claim 23, wherein a particle size of about 50 percents of the particles of the powdered fluticasone propionate ranges from about 3 microns to about 5 microns.

28. The process of claim 19, wherein said solvent is selected from the group consisting of an alcohol, a ketone and a mixture thereof.

29. The process of claim 28, wherein said alcohol is selected from the group consisting of ethanol and isopropanol.

30. The process of claim 28, wherein said ketone is selected from the group consisting of acetone and methyl ethyl ketone.

31. The process of claim 28, wherein a ratio between said alcohol and said ketone in said mixture is about 1:1.

32. The process of claim 19, wherein said spray drying is performed at an outlet temperature greater than 60° C.

33. The process of claim 32, wherein said spray drying is performed at a flow of about 50 m³/hour.

34. A powdered fluticasone propionate prepared by the process of claim 19.

35. A pharmaceutical composition being formulated for administration by inhalation, comprising the powdered fluticasone propionate of claim 34.

36. A powdered S-fluoromethyl-6α9α-difluoro-11β-hydroxy-16α-methyl-3-oxo-17α-propionyloxyandrosta-1,4-diene-17p-carbothioate (fluticasone propionate) being characterized by at least one characteristic selected from the group consisting of:
an average particle size that ranges from about 1 micron to about 10 micron;
free flowing;
a substantially spherical particles shape; and
a substantial absence of an electrostatic charge.

37. The powdered fluticasone propionate of claim 36, being substantially crystalline.

38. The powdered fluticasone propionate of claim 36, being partially amorphous.

39. The powdered fluticasone propionate of claim 36, wherein said average particle size ranges from about 1 micron to about 5 microns.

40. The powdered fluticasone propionate of claim 39, wherein a particle size of at least 90 percents of the particles of the powdered fluticasone propionate ranges from 1 to 5 microns.

41. The powdered fluticasone propionate of claim 37, wherein a particle size of about 50 percents of the particles of the powdered fluticasone propionate ranges from about 2 microns to about 3 microns.

42. The powdered fluticasone propionate of claim 38, wherein a particle size of about 50 percents of the particles of the powdered fluticasone propionate ranges from about 3 microns to about 5 microns.

43. A pharmaceutical composition being formulated for administration by inhalation, comprising, as an active ingredient, the powdered fluticasone propionate of claim 36.

44. A process of purifying 6α,9α-difluoro-11β-hydroxy-17α-propionyloxy-16α-methyl-pregna-3-oxo-1,4-diene-17α-thiocarboxylic acid, the process comprising:
providing 6α,9α-difluoro-11β-hydroxy-17α-propionyloxy-16α-methyl-pregna-3-oxo-1,4-diene-17β-thiocarboxylic acid;
providing a solution of said 6α,9α-difluoro-11β-hydroxy-17α-propionyloxy-16α-methyl-pregna-3-oxo-1,4-diene-17β-thiocarboxylic acid and an organic solvent;
contacting said solution with an aqueous solution containing a base, to thereby provide an aqueous solution containing a base addition salt of said 6α,9α-difluoro-11β-hydroxy-17α-propionyloxy-16α-methyl-pregna-3-oxo-1,4-diene-17β-thiocarboxylic acid;
isolating said aqueous solution containing said base addition salt;
converting said base addition salt into said 6α,9α-difluoro-11β-hydroxy-17α-propionyloxy-16α-methyl-pregna-3-oxo-1,4-diene-17β-thiocarboxylic acid; and
isolating said 6α,9α-difluoro-11β-hydroxy-17α-propionyloxy-16α-methyl-pregna-3-oxo-1,4-diene-17β-thiocarboxylic acid, to thereby provide a purified 6α,9α-difluoro-11β-hydroxy-17α-propionyloxy-16α-methyl-pregna-3-oxo-1,4-diene-17β-thiocarboxylic acid.
